(19) United States
(12) Patent Application Publication
(10) Pub. No.: US 2011/0021868 A1
(43) Pub. Date: Jan. 27, 2011

(54) Tissue Repair Device

(76) Inventor: James Browning, Glasgow (GB)

Correspondence Address: K&L Gates LLP, STATE STREET FINANCIAL CENTER, One Lincoln Street, BOSTON, MA, 02111-2950, US

(21) Appl. No.: 12/373,249

(22) PCT Filed: Jul. 11, 2007
(86) PCT No.: PCT/GB07/22589

§ 371 Date: Jan. 9, 2009

(30) Foreign Application Priority Data
Jul. 11, 2006 (GB) 0613653.5

(51) Int. Cl. A61F 2/00

(52) U.S. Cl. 600/37

(57) ABSTRACT

The present invention relates to a tissue repair device for use in surgical methods. The tissue repair device comprises a filiform having a longitudinal axis and a transverse width, wherein a force of at least 30 N can be exerted along the longitudinal axis of the device without distortion of the device and at least part of said filiform comprises interstices on a surface of the filiform, the interstices having a cross-sectional width in the range of 50 micrometers to 200 micrometers. In surgical methods, features of the device can provide for lubricious movement of the device within surrounding tissue for up to 72 hours, providing for adjustment of the device and positioning of the device in the body after its initial placement. Subsequently, traction of the device to the surrounding tissue can be achieved as the features of the device cause the device to act as a scaffold for tissue ingrowth into and around the structure of the device.

### FIELD OF INVENTION

This invention relates generally to a tissue repair device, which may be used, for example, in the field of surgery, In particular, the tissue repair device may be used in surgical methods of: supporting the urethra, repairing the pelvic floor, treating cystocoeles, treating rectocles or treating other prolapse conditions.

### BACKGROUND

Conventionally, in surgical methods, sutures have been used to pull two points together, for example, two separate portions of tissue or a portion of tissue and a portion of implant. However, sutures, due to their cord like structure, whilst providing high tensile strength, have not provided suitable structures for tissue ingrowth.

Conventional surgical meshes, comprising planar strand structures of connected polygons with space being provided between the strands to minimise the mass of the mesh, provide a scaffold for tissue ingrowth, but do not have sufficient strength to ensure that the meshes are not distorted or extended when force is applied along a longitundial axis of the mesh. In view of the lack of strength of conventional meshes such meshes cannot be used to pull two points together, for example two separate portions of tissue together.

Despite existing technology, there continues to exist a need in this art for novel tissue repair devices.

### SUMMARY OF INVENTION

The present inventor has surprisingly determined a surgical device which has both sufficient tensile strength such that it is capable of pulling two distinct points together, for example two portions of tissue, or a portion of tissue and an implant, or anchoring or securing implants in a suitable position in the body, and which can act as a tissue scaffold to encourage post operative tissue growth into and around the device when the device is implanted into a body.

Accordingly, a first aspect of the present invention provides a tissue repair device comprising a filiform having a longitudinal axis and a transverse width, wherein a force of at least 30 N can be exerted along the longitudinal axis of the device without distortion of the device and at least part of said filiform comprises interstices on a surface of the filiform, the interstices having a cross-sectional width in the range of 50 micrometres to 200 micrometres.

In embodiments of the device a force of at least 35 N, more preferably at least 40 N, more preferably at least 45 N, more preferably at least 50 N, more preferably at least 55 N, and yet more preferably at least 60 N can be exerted along a longitudinal axis of the device without distortion of the device.

In particular embodiments, a force of at least 70 N can be exerted along a longitudinal axis of the device without distortion of the device. 30 N can be considered to be the normal surgical force used. 70 N can be considered to be in the upper range of normal force used in typical surgical methods.

Distortion includes undesired twisting, pulling, elongation or tearing of the tissue repair device, as observed by the naked eye.

In embodiments of the device, distortion of the device can be an increase in the longitudinal length of the device along a longitudinal axis of more than 10% on application of a force along the longitudinal axis of the device.

In particular embodiments of the device, distortion of the device can be an increase in the longitudinal length of the device along the longitudinal axis of more than 5%, more than 2.5%, more than 2%, more than 1.5%, more than 1%, on application of a force along the longitudinal axis of the device.

Mechanical properties of a device can be determined using a procedure which follows the guidance specified in ISO 527:Tensile testing. The samples are held under tension in a force testor (Instron) so that the force applied to the sample gradually increases until the sample breaks. The tensile strength is the load at break while the elongation at break is extension of the sample at breaking point. The modulus of elasticity can be determined from a graphical plot of stress vs. strain over the elastic region of the curve. In one example of a suitable method for determining the mechanical properties of a device, the device to be tested is placed in jaws of the equipment and a series of measurements obtained which will detail values such as, for example, thickness of device, width of device, E modulus (MPa), Tensile Strength (Kg/cm²), Elongation to break (%). Determination of maximum force and elongation at maximum force for a mesh/textile, can use a strip method based on guidance as provided by ISO 13934-01.

Advantageously a device of the present invention provides the characteristics of a suture during insertion and in the first two to three days following insertion, for example high tensile strength and relatively low resistance to movement in tissue, and then provides for tissue ingrowth, which is presently only associated with meshes. Ingrowth of tissue results due to the acute inflammatory response of a body to surgical insult, (placement of the device). The features of the device, for example the interstices and the thickness of the device provides for tissue ingrowth into and through the device after a period of around 48 hours of placement of the device in a body, increasing the resistance to movement of the device, aiding fixation of the device within the body and the strength of support as provided by the device.

In use of embodiments of the device in surgical methods, the features of the device provide for lubricious movement of the device within surrounding tissue for up to 72 hours, providing for adjustment of the device and positioning of the device in the body after the initial placement of the device into the body. Subsequently, in embodiments of the device traction of the device to surrounding tissue is provided as the features of the device cause the device to act as a scaffold for tissue ingrowth into and around the structure of the device.

In particular embodiments the device can comprise substances or mechanical features which stimulate the inflammatory response of a body to the device.

In embodiments, said filiform can comprise at least one filament arranged to provide interstices of cross-sectional width in the range 50 micrometres to 200 micrometres. In particular embodiments at least two filaments are intertwined together to form a filiform with interstices in the range 50 micrometres to 200 micrometres. In such embodiments, intertwining can be achieved by any suitable means in the art, for example, by knitting, for example warp knitting, spinning, embroidery or the like.

In embodiments of the device wherein two filaments are intertwined, for example using a warp knit, the filaments can be arranged to provide strands with interstices in the range 50 micrometres to 200 micrometres between the filaments. In said embodiments, the strands of intertwined filaments can then be further intertwined together such that multiple strands are joined together wherein the spaces between the strands are in the range of 50 micrometres to 200 micrometres. The multiple strands joined together as described provides a filiform of desired width to provide the necessary strength characteristics and to act as a tissue scaffold.

In embodiments the entire surface of the filiform can comprise interstices of cross-section width in the range 50 micrometres to 200 micrometres.

Suitably, in particular embodiments, the tissue repair device may have a tensile breaking strength of at least 70, in specific embodiments at least 90 N, along its longitudinal length.

Suitably, in particular embodiments, the filiform may be 11 mm in width or less, 10 mm or less in width, 9 mm or less in width, 7 mm or less in width, more suitably less than 5 mm in width, even more suitably less than 3 mm in width.

In particular embodiments the filiform has a width in the range 1 mm to 6 mm. This is advantageous as passage of the tissue repair device through tissue of the body during placement of device is more easily achieved.

Preferably the filiform may be in the range 1 mm to 3 mm, most preferably 2 mm to 3 mm in width. In such embodiments when the device can have a first surface of a first width and a second opposite surface of a second width, wherein the first and second surfaces are spaced apart by the thickness of the device. A first width and second width may be equal.

Suitably, in embodiments of the device, the filiform can have a thickness in the range 10 to 50 micrometres. In particular embodiments the filiform can have a thickness in the range 20 to 35 micrometres. Functionally, in preferable embodiments, the device has a thickness, such that in use, fibroblasts of the body in which the device is inserted can enter the interstices of the device, and extend through the device from a first surface of the device to a second surface.

In embodiments of the tissue repair device, the device can be less than 900 mm in length, less than 800 mm in length, less than 700 mm in length, less than 600 mm in length, less than 500 mm in length, less than 400 mm in length, less than 300 mm in length, less than 250 mm in length, less than 200 mm in length, less than 100 mm in length, less than 50 mm in length, less than 25 mm in length. In particular embodiments of the device, at least one terminal end of the device may be a thickened or expanded portion of filiform. Optionally, such a thickened portion of the device is used to attach the device to tissue or another medical device, for example, an Implant. In embodiments of the device, at least one terminal end of the device may comprise a hole adapted to receive a polymer rivet, a suture or a staple.

In a preferred embodiment, the device can be a filiform of width in the range 2 mm to 11 mm, of thickness in the range 10 micrometre to 50 micrometres and at least one surface of the filiform can be provided with interstices of cross-sectional width in the range 50 micrometres to 200 micrometres.

It is advantageous to provide a filiform with as little width or thickness as possible, which provides suitable tensile strength to the device and minimises distortion of the device, such that in use, when the tissue repair device is provided in the body, the foreign body mass provided in the body is minimised.

In particular embodiments, the tissue repair device can be provided with a width and thickness such that, in use, the device can be moved through soft tissue during placement of the device and in the first two to three postoperative days with a force less than or equal to 50 N, less than or equal to 40 N, less than or equal to 30 N or less than or equal to 25 N.

The force required to move a tissue repair device may be determined using a suitable tissue model or tissue surrogate model, and a force gauge. Applying a constant rate of movement to a device to be tested, the device can be moved through the tissue model or tissue surrogate model and the force generated determined.

Suitably, in particular embodiments, said tissue repair device may be moved through soft tissue during placement of the device and in the first three postoperative days with a force less than or equal to 23 N.

In particular embodiments the tissue repair device may be moved through soft tissue during placement of the device and in the first three postoperative days with a force less than or equal to 20 N.

In particular embodiments the tissue repair device can include a connector portion, for example, but not limited to, a clip fastener, a hook, an aperture, a welded portion, or adhesive portion, to conjoin a tissue repair device to, for example, a support, or a suture.

In particular embodiments the device comprises a cross section which enables fibroblasts entering the interstices of the device to extend from a first surface of the device, through at least a part of the thickness of the device to a second surface of the device. The device can be planar, or another cross section, for example, but not limited a substantially circular cross section, an elliptical cross section, an ovoid cross section, a triangular cross section or a rectangular cross section.

In particularly preferred embodiments the cross section of the device is planar, and the device has a nominal thickness in one plane such that cells can move into the interstices of the device and extend from a first planar surface of the device, through the nominal thickness, to a second planar surface of the device.

Surface Structure

Suitably in embodiments of the device, the surface of the tissue repair device can be a substantially smooth surface, which provides little resistance to movement of the device within tissue. In particular embodiments the tissue repair device comprises at least one filament, preferably two filaments, intertwined wherein the largest space(s) between the intertwined filament(s) is of maximum cross-sectional width of 200 micrometres and a minimum cross-sectional width of 50 micrometres. A filament or filaments may be intertwined using suitable methods of the art, including knitting, spinning, embroidery, braiding or the like.

In particular embodiments the device can be provided with a plastic sleeve which covers at least a portion of the surface of the device during insertion of the device into the body. The plastic sleeve can advantageously provide the device with a smooth surface.

In particular embodiments, the device can comprise a coating or a film applied to at least part of the surface of the filiform, which when the device is in use and being inserted into tissue, reduces the friction between the device and the surrounding tissue. This is advantageous as the shear forces exerted on the device as it is moved within a body during insertion and subsequent placement of the tissue repair device are reduced. This minimises the risk of distortion or extension of the device.

In specific embodiments, a coating applied to a surface of the device can be a lubricious coating to provide the device with a substantially smooth surface and/or other characteristics which are desirable in surgical handling. A substantially smooth surface minimises the likelihood of damage to surfaces in the body which contact the device during insertion and placement of the device.

In particular embodiments, the lubricious coating provided to the device can be substantially absorbed by the body or can substantially degrade over a period of at least 24 hours, at least 48 hours, or up to 72 hours. Suitably, in embodiments, the lubricious coating or film can include, but is not limited to, proteins, polysaccharides, hydrophilic polymers, wax, hydrogel, silicone, silicone rubber, PTFE, PBA, ethyl cellulose or the like.

The lubricious coating may be disposed on the entire surface of the device or a portion thereof.

Advantageously, in embodiments of the device, the provision of a lubricious coating, which is absorbed by the body or degrades in the body over a pre-determined period of time, can provide a substantially smooth surface of the device during insertion and further provide the device with intrinsic lubricity, ideally for around two to three days following insertion of the device into the body. This facilitates insertion and manoeuvrability of the device during surgery and post operatively until tissue ingrowth occurs.

The coating can be applied as a film to the device. In suitable embodiments a film can contain polymers and/or compolymers of lactides, glycolides, caprolactone, triimethylene carbonate, or the like.

Typically, by around two to three days following insertion of the device into the body, tissue ingrowth into the device occurs and the device as provided no longer has sufficient lubricity to allow movement of the device through soft tissue with a force less than or equal to 20 N. In particular embodiments, in use, after at least two days, but less than a defined period, following insertion of the device in the body, the interstices of the device may allow sufficient tissue ingrowth to require a force of greater than or equal to 25 N, greater than or equal to 30 N, greater than or equal to 40 N, greater than or equal to 45 N to allow movement of the device through soft tissue.

Depending on the structural features of the device, for example, the number of interstices and/or the time taken for a coating or film provided to the device to degrade or be absorbed, the defined period may be selected from less than 3 days, less than 5 days, less than 10 days, less than 30 days, less than 60 days, less than 90 days, less than 6 months, or less than one year.

As will be appreciated by those of skill in the art, a substantially smooth surface may be provided during manufacture of an embodiment of the device using a variety of techniques as known in the art, for example, but not limited to using a close knit, or close weave to form the filiform. In particular embodiments a close warp knit can be used.

In particular embodiments, at least one filament, preferably a di-filament, can be arranged using a close warp knit to form a filiform and provide interstices in the surface of the filiform wherein the interstices of the warp knit have a cross-sectional width in the range 50 micrometres to 200 micrometres.

Embodiments of the device can be formed by a monofilament, a multifilament, or a mixture of monofilament and multifilament.

Alternatively, in particular embodiments, the device can be formed using non-woven or compression methods.

In particular embodiments the tissue repair device may comprise at least one projection which extends outwardly at an angle from a surface or edge of the tissue repair device.

Suitably, in particular embodiments of the device, in use, a projection may only allow the tissue repair device to move in the surrounding tissue in one direction with a force less than or equal to 30 N or that is sufficient to prevent distortion of the device. Additionally or alternatively, a projection(s) can provide the device with increased holding strength or traction in a tissue or against another part of the device or of an implant, for example the device can be used to secure a surgical mesh position around at least one anatomical structure to provide support to said structure. In particular embodiments, the increased holding strength or traction provided to the device to a tissue, the device, or an implant by a projection(s) is such that additional or alternative devices, for example sutures or fasteners, need not be used to join the device to tissue, itself or another implant. Further, in particular embodiments, the increased holding strength or traction provided by at least one projection can be advantageous as it can remove the need to tie knots with the tissue repair device to secure the tissue repair device or an implant or secondary device to tissue. This can be advantageous as it can reduce the time required to suitably position the tissue repair device or an implant or secondary device to a tissue.

Suitably, in particular embodiments, the angle at which the projection extends from a surface or an edge of the tissue repair device is less than 90°.

Projections may be suitably formed using a variety of methods known in the art including, for example, but not limited to, moulding, stamping, press forming, the application of portions to the body of the device by adhesive, or by having at least one, preferably a multiplicity of cut(s) or cut out portion(s) in a surface or surfaces and/or edge or edges of the device.

In particular embodiments the projections can be arranged such that they have a spacing in the range of 1 mm to 10 mm from each other.

As regards the geometry of the projections, this can be varied as can the length of the projections from the surface or edge of the device. In particular embodiments, the cutting angle and depth of cut can be varied such that the distance the projection extends form the body of the tissue repair device and the angle the projection extends from the body of the device can be adapted to the specific requirements required, for example to hold the device or provide traction to the device in a particular tissue, against itself or in relation to another device, for example an implant.

In suitable embodiments the projections can extend from 1 mm to 10 mm from the body of the device and the angle between the surface or an edge of the tissue repair device and the projection can be at least 15°, at least 20°, at least 30°, at least 45°, at least 60°, at least 70°, at least 80°, or less than 90°.

In particular embodiments wherein there is at least two, for example two, three, four, five, six, eight, ten, twelve or more projections present, a projection can, where an even number of projections are provided, be aligned across the device opposite a respective projection. Alternatively, where an even or odd number of projections are provided, the projections can be staggered across the device or positioned around the perimeter of and/or surfaces of the device.

In particular embodiments of the device, projections may only be provided on a portion(s) of the device. For example, the projections can be positioned along a predetermined portion or portions of the device, such as, but not limited to, one end portion or both end portions of the device.

Suitably, in particular embodiments, the projection(s) can be formed from shape memory polymer for example a polyurethane based polymer, such that the angle at which the projection(s) extends from the surface or edge of the device can be increased on exposure of the device to heat, or where suitable polymers are used, radiation, for example, but not limited to a change in light or pH. In other embodiments, a projection can be formed from any suitable material, for example a hydrogel.

Suitably the increase in angle can be from an angle between the surface or edge of the device and the projection in the range 0° to 5°, 0° to 10° or 0° to 20° to an angle of at least 15°, at least 20°, at least 30°, at least 45°, at least 60°, at least 70°, at least 80°, or less than 90° between the surface or an edge of the tissue repair device and the projection.

In particular embodiments of the device at least a second projection or second plurality of projections can be provided on the device wherein said second projection(s) extend outwardly from the body of the device at an angle greater than 90°. As will be appreciated, the at least second or second plurality of projections can be positioned at a predetermined portion or portions of the device.

In embodiments of the device, the at least one projection may be deflectable with a force less than or equal to a force of 30 N provided in a first direction and not deflectable when the same force is applied in an opposite direction. In use, movement of the device in a first direction may cause deflection of such a projection such that little resistance to movement of the device in a first direction in a tissue is provided by the projection, whilst the projection may not be deflected during movement of the device in a second opposite direction. The projection will therefore provide resistance to movement of the device in the tissue in one direction and thus provide for unidirectional movement of the tissue repair device when inserted in tissue.

Suitably, in particular embodiments, the tissue repair device may comprise at least one shaped projection extending at an angle from the surface or edge of the device, wherein, in use, the shaped projection does not substantially restrict the movement of the device in a first direction, but causes restriction of movement of the device in a second opposite direction.

Suitably, in particular embodiments, the shaped projection may be triangular wherein the base of the triangular shaped projection is provided such that it extends perpendicularly from the longitudinal axis of the device. In such an embodiment the base of the triangular shaped projection can be wider than the width of the tissue repair device at that point, but the thickness of the triangular shaped projection can be the same as the device. In such an embodiment the lateral projections from the device will provide for unidirectional movement of the device.

In another embodiment, the shaped projection can be a rounded protrusion extending from a surface of the device, but not extending beyond the edges of the device. In such an embodiment the thickness of the device will be greater where the protrusion extends from the surface than at a region of the device adjacent to such a protrusion. However, in such an embodiment the width of the device will not be increased at the protrusion. This can provide for a ratcheting movement of the device to be felt by a surgeon as the device, when in use, is pulled through layers of tissue, for example, the endopelvic fascia.

In further embodiments of the device, a projection can extend from a surface and an edge of the device. In such embodiments, the projection can be three dimensionally arranged around the filiform for example, but not limited to, a cone shaped projection, a spheroid projection, or an ovoid projection. Suitably, in embodiments of the device, projections can be three dimensionally shaped to provide, in use, for unidirectional movement and/or a ratcheting movement of the device through layers of tissue. For example, the device may be able to move forward or backwards in a tissue by a defined distance, the distance being defined by the distance between a first projection and an adjacent projection.

Interstices

Whilst not wishing to be bound by theory, the inventor believes that the interstices of width in the range of 50 micrometres to 200 micrometres allow tissue ingrowth into the tissue repair device to supplement the fixation of the tissue repair device in the body and the strength of support provided by the tissue repair device.

Interstices include pores, pits or slits. A pore can be a passage which extends through the device from a first surface to a second surface, wherein said pore has a diameter in the range 50 μm to 200 μm. A pit is a depression or aperture in a surface of the device which does not extend through the device. A slit can be a passage which extends through the device from a first aperture to a second aperture, wherein the cross-sectional width of the first and/or second aperture in one direction is not equal to the cross-sectional width of the first and/or second aperture in a second direction. Further, the cross-sectional widths of the first and second apertures can differ from each other.

In particular embodiments, the device may comprise interstices in the range of 50 to 120 micrometres in cross-sectional width.

Suitably, in particular embodiments, the interstices may be in the range of 70 to 100 micrometres in cross-sectional width.

In particular embodiments, the interstices may be in the range of 50 μm to 75 μm in cross-sectional width.

Where the Interstices are circular, the width is a diameter.

Suitably, in particular embodiments, the tissue repair device may be formed from at least one filament. Where the filiform is formed from a single filament, the filament may be woven, knotted, braided or knitted to produce interstices in the filiform.

Alternatively, the filiform may comprise at least two filaments which may woven, knotted, knitted or braided together wherein said interstices are provided between the filaments.

Interstices may alternatively or additionally be provided by pores, pits or slits provided on the surface of the filiform.

Pores or slits provided on the surface of the filiform may extend through the filiform or alternatively extend into a central portion of the filiform. Pores may be formed using a variety of techniques known to those in the art, for example, knitting, weaving, knotting or post synthesis modification, for example, by a laser, mechanical or ultrasound “drilling”.

Grooves

It may be advantageous to encourage collagen fibre laydown in an ordered direction to promote the formation of at least one strong ordered neoligament. The formation of at least one ordered neoligament should advantageously add mechanical strength to tissue which forms around the tissue repair device.

Suitably, in particular embodiments, the tissue repair device may be provided with at least one microgroove on at least one surface of the tissue repair device. Advantageously, the provision of at least one microgroove encourages collagen laydown. A plurality of microgrooves may be provided on at least one surface of the tissue repair device.

Preferably a microgroove can be of width between 0.5 μm to 7 μm and of depth 0.25 μm to 7 μm. More preferably a microgroove can be 5 μm in width and 5 μm in depth. Suitably a plurality of microgrooves may be aligned such that they are substantially parallel with each other.

Preferably the plurality of microgrooves may be aligned such that they are separated by ridges which range in size between 1 μm to 5 μm in width. More preferably the microgrooves may be separated by ridges of 5 μm in width.

Suitably the ridges may be formed by square pillars and the base of the microgroove can be substantially perpendicular to the square pillars. Alternatively the ridges may be formed by square pillars and the base of the microgroove can be bevelled in relation to the pillars.

Materials and Methods of Manufacture

One skilled in the art will appreciate that the selection of a suitable material for forming the tissue repair device of the present invention depends on several factors, for example, but not limited to in vivo mechanical performance; cell response to the material in terms of cell attachment, proliferation, migration and differentiation; biocompatibility; and optionally, bioabsorption (or bio-degradation) kinetics.

Examples of material that can be suitable for use in the present invention include, but are not limited to, absorbable, non-absorbable, braided, monofilament, or multifilament material.

The device of the present invention can be preferably formed from a biocompatible synthetic material or a biocompatible natural material. In particular embodiments a biocompatible material may be selected from a synthetic polymer or synthetic polymers, a natural polymer or natural polymers or combinations thereof.

Suitably, in particular embodiments, biocompatible synthetic polymers may advantageously include polymers selected from the group comprising: polyester, polypropylene, lactide, polyglycolactide, polycaprolactone and blends and copolymers thereof.

Alternatively or additionally, in particular embodiments, the device can comprise a shape memory polymer, for example a polyurethane based polymer.

In embodiments, the tissue repair device may be formed completely or partially from absorbable material and/or polymer. Forming of a tissue repair device from absorbable material and/or polymer may be advantageous, as with time the device would be absorbed by the body and reduce the overall implant mass retained by the body. This would minimise the likelihood of prolonged inflammation against and rejection of the device and any implant attached thereto by the body.

In suitable embodiments, an absorbable material can comprise natural proteins and or natural biopolymers.

In particular embodiments absorbable polymers can be selected from glycolide, glycolic acid, lactide, lactic acid, ε-caprolactone, ρ-dioxanone, trimethylene carbonate, polyethylene glycolide, polyanhydride, polyhydroxyalkanoate and the like.

In other embodiments, the material may be formed from filaments comprising polyester polymers, polypropylene polymers and the like, copolymers, or blends thereof.

Suitably, in particular embodiments, the tissue repair device may be obtained by spinning, embroidery, weaving, knitting, braiding, or by non-woven techniques, for example, fibre bonding, air-laying, wet laying, extrusion and/or laminating fibres.

Non-woven fabrics, include, but are not limited to, bonded fabrics, formed fabrics, or engineered fabrics that are manufactured by methods other than spinning, weaving or knitting. In embodiments, the device may be formed from a flat sheet of non-woven or woven material.

In one embodiment a tissue repair device may be manufactured using woven techniques wherein said woven techniques are selected from the group comprising; knitting, spinning, braiding, weaving or embroidery. In such embodiments, at least one filament can be woven to provide the filiform with interstices on a surface of the filiform, the interstices having a cross-sectional width in the range of 50 micrometres to 200 micrometres.

In other embodiments a tissue repair device may be manufactured using non woven techniques wherein said non-woven techniques are selected from the group comprising; fibre bonding, air-laying, wet laying, extrusion, compression or laminating. In such embodiments, interstices on a surface of the filiform having a cross-sectional width in the range of 50 micrometres to 200 micrometres can be provided as part of the manufacturing process or subsequence to the manufacturing process.

In a particular embodiment of the tissue repair device the filiform may be provided by warp knitting such that a filiform of width of 1 to 3 mm with a tensile breaking strength of at least 70 N is provided wherein said filiform comprises intertwined strands, the strands formed from intertwined filaments, wherein interstices between intertwined filaments and interstices between adjacent strands are in the range of 50 micrometres to 200 micrometres.

Bioactive Agent

Suitably, in particular embodiments, the tissue repair device may comprise at least one bioactive substance.

Bioactive substances include, but are not limited to, enzymes, proteins, peptides, either naturally occurring, recombinant or synthetic, pharmacological agent including, but not limited to; growth factors and wound healing agents.

Suitably, in particular embodiments, the bioactive substance may stimulate cell growth, promote healing and/or tissue repair. For example, the bioactive substance may be platelet rich plasma, cartilage-derived morphogenic proteins, recombinant human growth factors, chitosan, silver compounds and the like and combinations thereof.

Bioactive substance can be applied to the device during or post manufacture of the device.

As will be appreciated by those of skill in the art, by including a bioactive substance effective to stimulate cell growth, promote healing and/or tissue repair, tissue ingrowth into the tissue repair device may be accelerated.

Alternatively, or additionally bioactive substances may include compounds or agents that prevent infection (e.g., antimicrobial agents, antifungal agents, antiviral agents, and antibiotics), compounds or agents that reduce inflammation (e.g., anti-inflammatory agents), agents that suppress the immune system (e.g., immunosuppressants), local anaesthetics, pain relief substances, hormones or compounds that prevent or minimize adhesion formation.

It is understood that one or more bioactive agents of the same or different functionality may be incorporated within the device.

It will be appreciated that the Identity of the bioactive agent may be determined by a surgeon, based on principles of medical science and the applicable treatment objectives.

The amount of the bioactive agent included with the tissue repair device may vary depending on a variety of factors, for example the material from which the device is made, the identity of the agent, and the intended purpose of the tissue repair device. One skilled in the art can readily determine the appropriate quantity of bioactive agent to include for a given application in order to facilitate and/or expedite the healing of tissue. Suitably in embodiments of the device, bioactive substance(s) may be incorporated within and/or applied to the tissue repair device. Suitably In embodiments of the tissue repair device, the device may be provided with a harmless water soluble dye, for example methylene blue or another water soluble chemical capable of analytical detection. In particular embodiments the water soluble chemical capable of detection can be incorporated in a bodily fluid subsequently expelled from the body. The provision of said dye or agent may be advantageous where, in use, the device is located in proximity to the bladder. Following location of the device in the body a small amount of fluid may be expelled from the bladder. Should any of this fluid contain the agent or dye, for example methylene blue, it is likely that the bladder has been perforated on placement of the device.

Implants Including the Tissue Repair Device and Support Portion

At least one tissue repair device of the first aspect of the invention may further comprise a support portion, conjoined to the tissue repair device. Typically the support portion can be of greater width than the tissue repair device and need not have the same structural characteristics.

Accordingly, a second aspect of the present invention provides an implant comprising at least one tissue repair device and a support portion.

Suitably, in particular embodiments, the support portion can be provided by tape or mesh. In particularly preferred embodiments, the support portion can be comprised of the same materials as the tissue repair device, but be formed from a weave or knit of filaments such that spaces between strands formed by the filaments can have bigger dimensions, for example spaces with a cross-sectional width in the range 1 mm to 10 mm. In such embodiments, some spaces between filaments of the support portion may still have a cross-sectional width in the region of 50 micrometres to 200 micrometres.

Suitably in particular embodiments, where required, pairs of at least 2, at least 4, at least 6 tissue repair devices of the first aspect of the invention may be conjoined to a support tape portion or support mesh portion.

In particular embodiments wherein the support is a mesh the mesh may comprise spaces between the,strands of around 1 to 10 mm.

A mesh with strands spaced apart by 1 to 10 mm is advantageous as the overall mass of the support portion of the tissue repair device may be reduced.

Suitably, in particular embodiments the strands can be spaced apart to form spaces between the strands in the range 1.5 mm to 8 mm.

Suitably, in particular embodiments the strands can be spaced apart to form spaces between the strands in the range 1.8 to 2 mm.

Suitably, in particular embodiments the strands of the mesh can have a diameter of less than 600 micrometres.

The strands may be arranged in suitable net meshes which provide sufficient strength and elasticity to the support portion. For example, the net meshes may be, but not limited to, diamond or hexagonal net meshes.

In particular embodiments the mesh may be provided by warp knitting.

Suitably, in embodiments of the invention, the support portion may have a mass density of less than 30 grams per metre squared (g/m²), more preferably, less than 25 g/m², yet more preferably less than 20 g/m², yet more preferably less than 15 g/m², more preferably less than 10 g/m².

The provision of a support portion with a mass density of less than 30 g/m² is advantageous as it reduces all the risks of foreign body implantation around the tissue repair device when, in use, it is located in the body.

In embodiments of the tissue repair device comprising a support portion wherein said support portion is a mesh, the strands of the mesh of the support portion may be provided with interstices within the strands wherein the cross-sectional width of the interstices is in the range of around 50 micrometres to 200 micrometres. Suitably, in particular embodiments, the interstices may be 50 micrometres to 100 micrometres in cross-sectional width. In embodiments, the interstices may be 50 micrometres to 75 micrometres in cross-sectional width. As described above, the interstices can comprise a pore, slit or pit.

As detailed above, pores of around 50 micrometres to 200 micrometres in cross-sectional width are advantageous in promoting tissue ingrowth into the support portion.

Suitably in embodiments, a support portion may be conjoined to the at least one tissue repair device by any available fixing means. For example, fixing means include, but are not limited to, a suture(s), a rivet(s), for example a polymer rivet(s), a staple(s), glue, or the support portion may be conjoined by knitting.

In particular embodiments wherein the tissue repair device comprises a projection, a support may be fixed to a tissue repair device by the projection.

To aid the joining of at least one tissue repair device to a support portion either one or both of the tissue repair device or support portion may be provided with terminal expanded portion.

Advantageously such a terminal expanded portion may be smoothed at a surface such that, for example, a filament or filaments of the filiform of the tissue repair device and/or the support portion do not project from the device or support or the filament(s) do not unravel.

Suitably, one or both of the tissue repair device or support portion may be provided with a hole which extends through the thickness or width of a device or support which allows the passage of fixing means through said device and/or support.

According to a third aspect of the present invention there is provided a kit comprising
a tissue repair device comprising a filiform having a longitudinal axis and a transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device.

; and

a needle introducer.

Preferably a force of at least 35 N, at least 40 N, at least 45 N, at least 50 N, at least 55 N, at least 60 N or at least 70 N can be exerted along the length of the device without distortion of the device.

Preferably the needle introducer can be a colposuspension type needle with a malleable shaft and sharp tip. In particular embodiments the needle introducer can be a Nottingham needle™.

Suitably in particular embodiments the kit can further comprise a support portion. In specific embodiments the support portion can be a mesh or a tape.

Suitably, in particular embodiments, the kit may comprise fixing means, for example, but not limited to a suture(s), a rivet(s), for example a polymer rivet(s), glue, or a staple(s), for fixing the support portion to the tissue repair device.

In use, the tissue repair device may exit the body through the skin. In these circumstances, the tissue repair device may be held in position at the skin by a button or the like or a suture.

Suitably, in particular embodiments, the kit may comprise at least one button for fixation of the tissue repair device at the skin.

When, in use, the tissue repair device of such embodiments can be held in position at the skin by the button or suture. It may be advantageous to provide a collar device around the button or suture to retain, for example, the button below the surface of the skin. Suitably the kit may comprise a collar device wherein the collar device includes a depression arranged to receive the button wherein said collar device has a passage extending therethrough, which can accommodate a portion of the tissue repair device.

In particular embodiments, the collar device may include a radial cutout to allow its positioning around the tissue repair device when the tissue repair device is located in the body. In use, the collar device can be located around the tissue repair device above the skin surface, the button can be located on the tissue repair device and moved towards the collar device such that the button is moved into the depression of the collar device. This movement can push the collar device into the skin surface, without breaking the surface of the skin such that the depression provided in the collar device is lower that the surface of the surrounding skin and thus the button in the depression is lower than the surrounding skin.

In particular embodiments, the collar device may include means to allow only unidirectional movement of the tissue repair device through the collar. This may be advantageous to allow successive movement through and holding of the tissue repair device over a period of time.

In particular embodiments when the needle introducer has an aperture adapted to receive an end of the tissue repair device, an end of the tissue repair device, which can be held by a needle introducer, can be tapered to a point to ease the passage of the tissue repair device into the aperture of the needle introducer.

The inventor has surprisingly determined that a tissue repair device of the present invention advantageously allows operative and postoperative adjustment of the tissue repair device and then subsequently allows tissue ingrowth into the tissue repair device.

In particular embodiments of the tissue repair device wherein the device is conjoined to the needle introducer, the device can be attached to the needle introducer at a portion of the device with reduced width and/or thickness such that the force required to separate the needle introducer from the device is less than the tensile strength of the device.

Advantageously tissue ingrowth increases the strength of attachment provided by the tissue repair device to surrounding tissue.

The tissue repair device of the first aspect of the present invention, and/or the kit of the second aspect of the present invention can be used in many types of surgical procedures. For example, the device and/or kit can be used in surgical procedures, including, but not limited to, general procedures, gynaecological procedures, cardiovascular procedures, peripheral-vascular procedures or cardio-thoracic procedures.

In particular, the tissue repair device can be used to attach medical devices for example, but not limited to a prosthesis or implant, to tissue, tissue to tissue, or a first medical device, for example a prosthesis or implant to a second medical device.

According to a fourth aspect of the present invention there is provided a method of connecting a first fixing point to a second fixing point, comprising:

providing a tissue repair device comprising a filiform having a longitudinal axis and a transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device; and

fixing a first end of the device to the first fixing point; and

fixing a second end of the device to the second fixing point.

In particular embodiments the first and second:fixing points are first and second portions of tissue. In embodiments at least one of the first and second fixing points are tissue. In embodiments at least one of the first and second fixing point can be a medical device, a fastener outside the body, a suture(s), or implant or the like such that the tissue repair device extends for example between a tissue portion and an implant or between a first implant and a suture.

In particular embodiments of the method, the method can comprise a step of adjusting the position of the device within the body. In particular embodiments, adjustment of the position of the tissue repair device held between the first and second fixing points can be performed within 72 hours from insertion of the device into the body. In particular embodiments, the step of adjusting can occur after 2 hours following insertion of the device into the body, after 4 hours following insertion of the device into the body, after 8 hours following insertion of the device into the body, after 16 hours following insertion of the device into the body, after 24 hours following insertion of the device into the body. As previously outlined above, the features of the device of the present invention provide for tissue ingrowth following around 72 hours and until tissue ingrowth the device can have a substantially smooth and/or lubricious surface to allow movement through the surrounding tissue.

According to a fifth aspect of the present invention there is provided a method of supporting a defined tissue structure in the body comprising the steps of,

providing a tissue repair device comprising a filiform having a longitudinal axis and a transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device;

fixing a first end of the device to a first fixing point on a first side of the defined tissue structure to be supported;

fixing a second end of the device to a second fixing point on a second side of the defined tissue structure to be supported; and

positioning the device under the defined tissue structure to be supported.

In particular embodiments the first and second fixing points are first and second portions of tissue. In embodiments at least one of the first and second fixing points are tissue. In embodiments at least one of the first and second fixing point can be a medical device, a fastener outside the body, a suture(s), or implant or the like such that the tissue repair device extends for example between a tissue portion and an implant or between a first implant and a suture.

In particular embodiments of the method, the method can comprise the step of adjusting the position of the device within the body. In particular embodiments, the position of the tissue repair device held between the first and second fixing points can be adjusted within 72 hours following insertion of the device into a body. In particular embodiments, the step of adjusting can occur after 2 hours following insertion of the device into the body, after 4 hours following insertion of the device into the body, after 8 hours following insertion of the device into the body, after 16 hours following insertion of the device into the body, after 24 hours following insertion of the device into the body. As previously outlined above, the features of the device of the present invention provide for tissue ingrowth following around 72 hours and until tissue ingrowth the device can have a substantially smooth and/or lubricious surface to allow movement through the surrounding tissue, for example to suitable position the device under the defined tissue structure, for example, but not limited to an anatomical structure or organ to be supported.

In particular embodiments, the tissue repair device can comprise a support portion interposed between the first and second ends of the device. In particular embodiments, the support portion can have a width in the range 8 to 15 mm preferably about 11 mm and a length of about 80 mm.

In particular embodiments of the method of the fifth aspect of the present invention, the tissue repair device can be used to support the urethra.

According to a sixth aspect of the present invention there is provided a method of supporting a defined tissue structure in the body comprising the steps of,
providing a first and second tissue repair device comprising a filiform having a longitudinal axis and transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device and a support portion interposed between said first and second devices, wherein said support portion is conjoined to one end of each of the first and second devices,
inserting a first end of a first tissue repair device through an incision in a body, past a first lateral side of a defined structure to be supported such that that at least part of the first end of the first tissue repair device exits the body;
inserting a first end of the second tissue repair device through an incision in the body, past a second lateral side of the defined structure to be supported such that at least part of the first end of a second tissue repair device exits the body; and
positioning the support portion interposed between the first and second tissue repair devices adjacent to the defined tissue to be supported such that the support portion provides support to the defined tissue.

In one embodiment of the sixth aspect of the present invention the method of supporting a defined tissue structure in the body, is a method of supporting the urethra comprising the steps of,
providing a first and second tissue repair device comprising a filiform having a longitudinal axis and transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device and a support portion interposed between said first and second devices, wherein said support portion is conjoined to one end of each of the first and second devices,
inserting a first end of a first tissue repair device through a vaginal incision, past a first lateral side of a urethra and through an abdominal wall such that at least part of the first end of the first tissue repair device exits the body;
inserting a first end of the second tissue repair device through a vaginal incision, past a second lateral side of a urethra and through an abdominal wall such that at least part of the first end of the second tissue repair device exits the body; and
positioning the support portion under the urethra to provide support to the urethra.

In particular embodiments, each of the first and second devices can have a width in the range 2-3 mm and can be around 200 mm to 400 mm in length. In suitable embodiments, a support portion can be around 8 mm to 15 mm in width and of length 40 mm to 120 mm, preferably 80 mm in length.

To minimise the risk of bladder or bowel perforation, instead of extending through the abdominal wall the tissue repair device of the present invention may also be positioned in the body such that it exits the body via the obturator foramen.

In another embodiment of the sixth aspect of the invention there is provided a method of supporting a urethra comprising the steps of,
providing a first and second tissue repair device comprising a filiform having a longitudinal axis and transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device and a support portion interposed between said first and second devices, wherein said support portion is conjoined to one end of each of the first and second devices,
inserting a first end of the tissue repair device through a vaginal incision, past a first lateral side of a urethra and through a first obturator foramen such that at least part of the tissue repair device exits the body;
inserting a first end of a second tissue repair device through a vaginal incision, past a second lateral side of a urethra and through a second obturator foramen such that at least part of the tissue repair device exits the body; and
positioning the support portion under the urethra to provide support to the urethra.

Preferably a method of the invention may further comprise the step of adjusting the position of at least a first and/or second tissue repair device or support portion. Suitably, in particular embodiments, such adjustment occurs within 72 hours of the initial placement of the device in the body. In particular embodiments, the step of adjusting can occur after 2 hours following insertion of the device into the body, after 4 hours following insertion of the device into the body, after 8 hours following insertion of the device into the body, after 16 hours following insertion of the device into the body, after 24 hours following insertion of the device into the body. In suitable embodiments, the step of adjusting can include pulling on at least one end of a tissue repair device to adjust the position of the support portion relative to the tissue/anatomical structure to be supported, for example the urethra, following insertion of the device. Suitably in particular embodiments of the method the step of adjusting may take place in a period following insertion greater than one hour following insertion and less than three days following insertion.

Advantageously this provides for the position of the support portion in the body for example, under the urethra, to be altered/improved following initial placement. Advantageously, the patient may be allowed to recover from the operation, for example from any anaesthetic which has been provided, and the location of the support portion and tissue repair device can be modified following discussion with the patient to minimise discomfort to the patient whilst maximising performance of the support portion to support to an anatomical structure, for example, the urethra. The interstices of the device, which provide for tissue ingrowth into the device in response to the acute inflammatory response of the body against the tissue repair device, provide for the support and strength of the device after two to three days post insertion of the device into the body.

Due to the reduced width of tissue repair device of the present invention as opposed to conventional meshes used to support anatomical structures and provide for tissue ingrowth, the tissue repair device of the present invention may be inserted into the body using a needle(s) of significantly less diameter than the insertion tools used in current procedures of providing supporting meshes and implants, for example sling procedures, as the dimensions of the tissue repair device require less force to be exerted to move the device through tissue. For example, a Nottingham needle™ may be used to insert a tissue repair device into the body and direct the same to the exit point of the body. In view of the reduced diameter of the needles which may be used in the methods of the present invention, the needles may be pushed out of the body without requiring incisions in the lower abdominal wall or over the obturator foramen. This may result in less trauma to the patient.

Accordingly, in preferred methods of the invention there is no requirement for incisions to be made in either the abdominal wall or above the obturator foramen to allow the implant to exit the body.

According to a seventh aspect of the present invention there is provided a method for supporting prolapse in at least one of vagina and uterus, comprising the steps:
providing at least a first pair and a second pair of tissue repair devices each device comprising a filiform having a longitudinal axis and transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device and a support portion interposed between said first and second pairs of devices, wherein said support portion is conjoined to one end of each of the first and second pairs of devices,
inserting a first end of a first pair of tissue repair devices through a vaginal incision, and through at least one of:
   an obturator foramen such that at least part of the tissue repair device exits the body;
   an abdominal wall such that at least part of the first end of the first tissue repair device exits the body; or
   perineal skin such that at least part of the tissue repair device exits the body;
inserting a first end of a second pair of tissue repair devices through a vaginal incision, and through at least one of:
   an obturator foramen such that at least part of the tissue repair device exits the body;
   an abdominal wall such that at least part of the first end of the first tissue repair device exits the body; or
   perineal skin such that at least part of the tissue repair device exits the body;
positioning the support portion to provide support to at least one of a vaginal wall and a uterus.

In particular embodiments the method of supporting prolapse further comprises a step of:
adjusting the position of the support portion to provide support to at least one of a vaginal wall and a uterus.

In specific embodiments the step of adjusting the position of the support portion is performed within 72 hours of inserting the device in the body. In embodiments, the step of adjusting can occur after 2 hours following insertion of the device into the body, after 4 hours following insertion of the device into the body, after 8 hours following insertion of the device into the body, after 16 hours following insertion of the device into the body, after 24 hours following insertion of the device into the body.

Suitably embodiments of the device and methods of the invention can be used to treat urinary incontinence.

Suitably embodiments of the device and methods of the invention can be used to repair a pelvic floor.

Suitably embodiments of the device and methods of the invention can be used for treatment of cystoceles, rectoceles and/or vault prolapse/enterocele.

Suitably embodiments of the device and methods of the invention can be used for treatment of prolapse, in particular uterovaginal prolapse.

Suitably embodiments of the device and methods of the invention can be used for treatment of male urinary incontinence.

Suitably embodiments of the device and methods of the invention can used for treatment of rectoanal sphincter disorders.

Preferred features and embodiments of each aspect of the invention are as for each of the other aspects mutatis mutandis unless context demands otherwise.

Embodiments of the present invention are described by way of example only with reference to the accompanying figures in which:

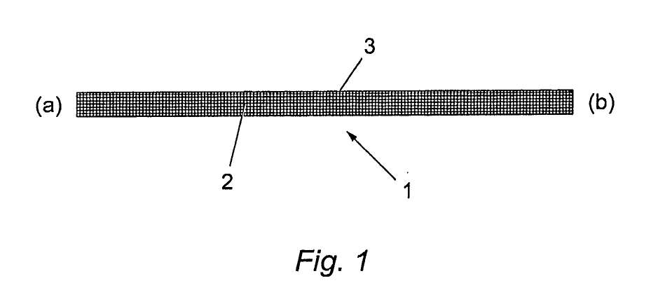

FIG. 1 is an illustration of an embodiment of a tissue repair device of the invention;

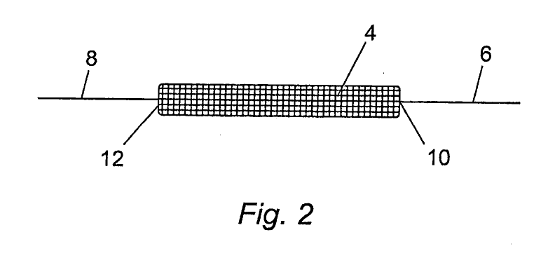

FIG. 2 is an illustration of an implant comprising a support portion conjoined to two tissue repair devices;

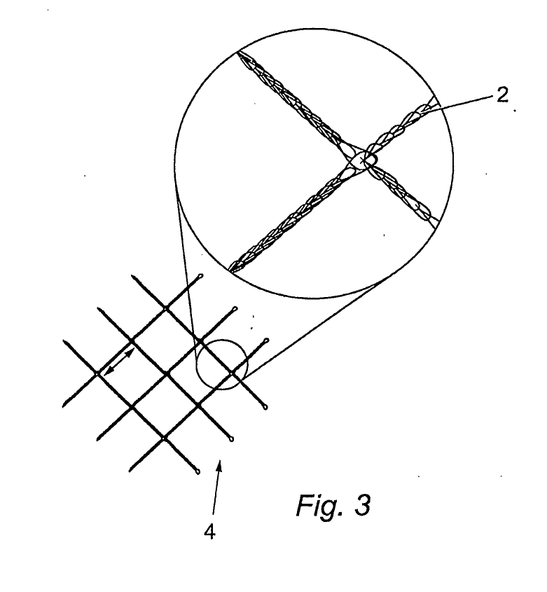

FIG. 3 is an illustration of a support portion when provided as a mesh comprising strands which define large spaces and include pores therein;

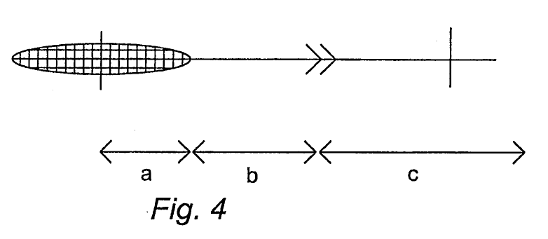

FIG. 4 is an illustration of an implant which may be used to correct urinary incontinence;

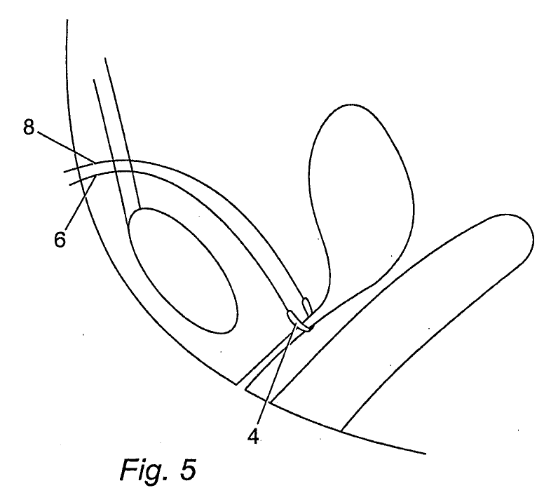

FIG. 5 is an illustration of an embodiment of a support portion conjoined to two tissue repair devices implanted in the body using a retropubic procedure;

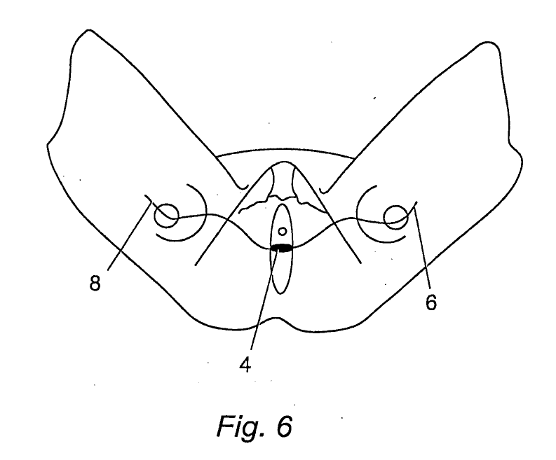

FIG. 6 is an illustration of an embodiment of a support portion conjoined to two tissue repair devices implanted in the body using an obturator foramen procedure;

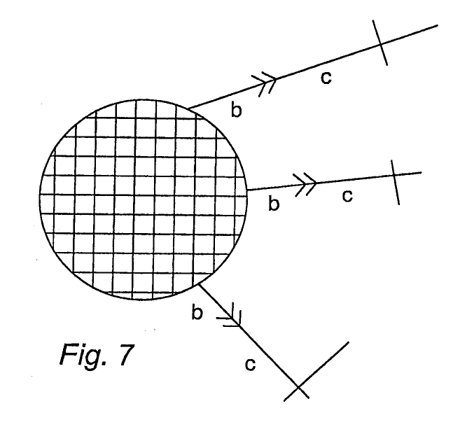

FIG. 7 is an illustration of an implant which may be used in the weakness of prolapse;

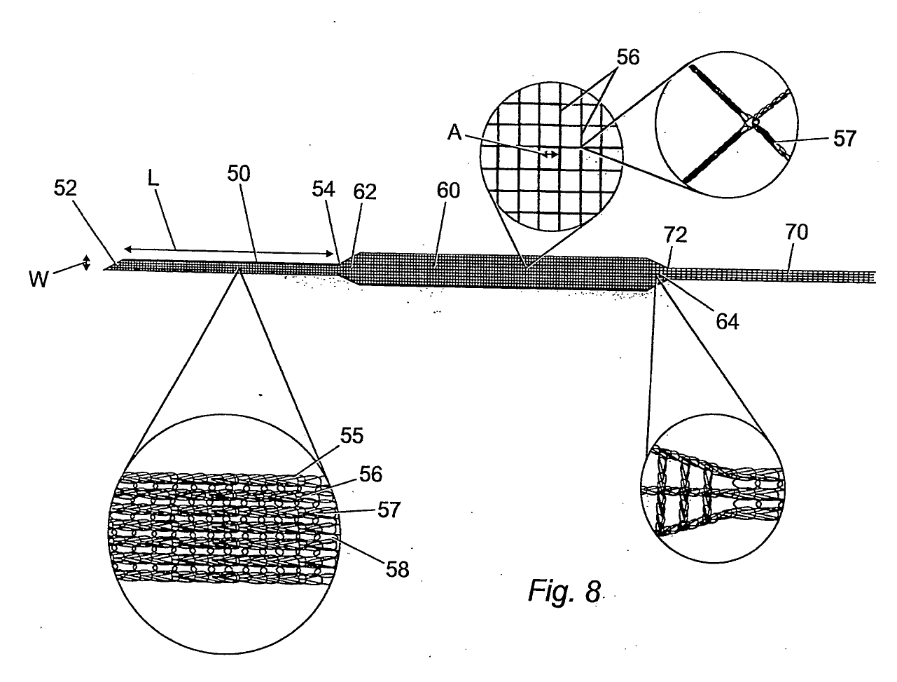

FIG. 8 is an illustration of an embodiment of two tissue repair device with a support portion interposed therebetween; and

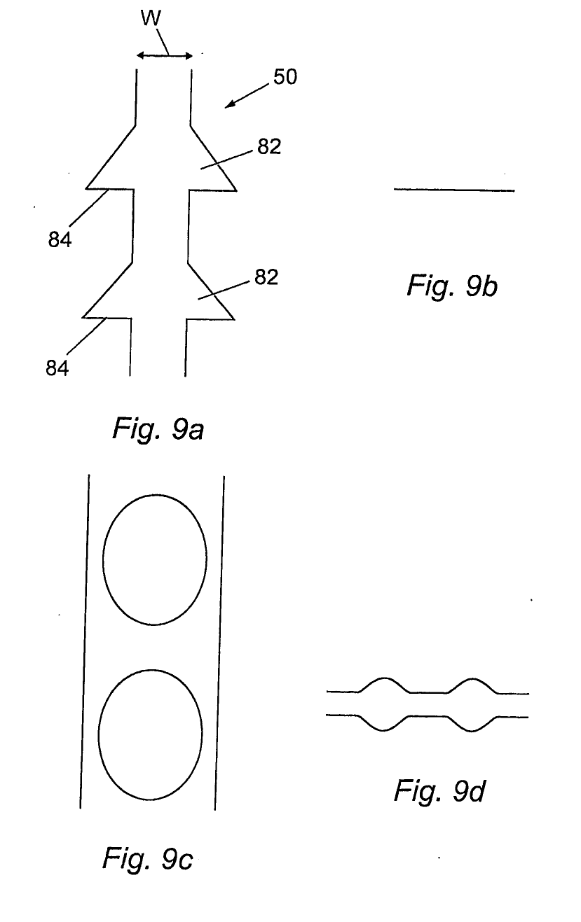

FIG. 9 illustrates projections present on a tissue repair device (a) and (b) are illustrations of a triangular projection in plan and cross-section respectively, and FIGS. 9(c) and (d) are illustrations of a rounded projection on a tissue repair device in plan and cross-section respectively.

### EXAMPLES

Tissue Repair Device

As shown in FIG. 1 in one embodiment the tissue repair device is a filiform 1 comprising interstices 2 having a cross-sectional width in the range of 50 micrometres to 200 micrometres. The filiform 1 has a longitudinal axis between (a) and (b) wherein a force of at least 30 N can be exerted along the longitudinal axis of the device without distortion of the device. In the embodiment shown the device has a tensile breaking strength of 70 to 90 N. The interstices are provided by the intertwining of multifilaments to form thicker strands and then intertwining of said strands to form the filiform. The intertwining is performed using a warp knit used to manufacture the device.

The tissue repair device has a substantially smooth surface 3 provided for by the warp knit and the lubricious coating applied to the device. This provides the device with intrinsic lubricity, for around two to three days following insertion of the device into the body. This facilitates insertion and manoeuvrability of the device during surgery and post operatively until tissue ingrowth into the interstices occurs.

Tissue Repair Devices with Interposed Support Portion

As illustrated in FIG. 2, an implant can be provided wherein the implant comprises a short segment of a mesh support portion 4 interposed between a first tissue repair device of the present invention 6 and a second tissue repair device of the present invention 8. Although the implant is described in detail below in relation to its use to support the urethra to treat urinary incontinence, the skilled person will understand that implants including the features described could be used to support any suitable anatomical structure or defined tissue, for example, but not limited to repair a pelvic floor, for treatment of cystoceles and rectoceles and prolapse conditions.

In use, as illustrated in FIG. 5, the support portion 4 of the implant 2 is positioned under the urethra.

In the embodiment of the implant illustrated in FIG. 2, suitable for treatment of urinary incontinence, the support portion 4 is around 4-8 cm In length and 0.5 cm to 1.2 cm in width.

As shown, a first device 6 extends from a first end 10 of the support portion and a second device 8 extends from a second end 12 of the support portion.

The tissue repair devices in this example are each of around 20 cm in length and around 2 mm in width.

In this example the tissue repair devices are substantially ovoid in cross section. This is advantageous, as it minimises the likelihood of damage to surrounding tissue by the device during insertion, post-operative adjustment and beyond.

However, those skilled in the art will appreciate that devices of other cross sections may be used provided that the surface of the device allows its Movement within the patient during implantation and for around two days post implantation without trauma to the tissues surrounding the device.

The implant as described above may be used to correct urinary incontinence, as shown in FIG. 5 or 6.

FIG. 4 shows an implant comprising non-absorbable and absorbable sections. The non-absorbable section from the midpoint of the mesh support portion and the tissue repair device (sections a and b) is around 4 to 14 cm and the absorbable portion of the tissue repair device, made from an absorbable polymer, (section c) is around 2 to 25 cm.

FIG. 5 shows an implant as described implanted in the body using a retropubic procedure.

In such a procedure a needle introducer is used to push the tissue repair device into, through and then out of the body. In particular embodiments the needle introducer is provided with an aperture through which a first end of the device may be passed. The friction between the edge of the aperture in the needle introducer and the device should retain the device in the aperture during the insertion of the device and the needle introducer into, through and out of the body. The end of a tissue repair device of the invention may be suitably tapered to facilitate the threading of the aperture with the device (tapering of ends of a tissue repair device for insertion into an aperture of a Nottingham needle is illustrated in FIG. 7).

As illustrated in FIG. 5, an incision is made in the vaginal wall, and the first of the surgical instruments, such as a Nottingham needle, to which the first device is attached is passed through the incision, past one side of the urethra, behind the pubic bone, through the rectus sheath and out through the lower anterior abdominal wall. Likewise, the second needle is passed through the vaginal incision, past the other side of the urethra, behind the pubic bone, through the rectus sheath and out through the lower abdominal wall. As the needle introducer is of a small diameter, no cutateous incision is required in the abdominal wall and the introducer may be merely pushed through the abdominal wall and create only a small puncture wound. The first and second tissue repair devices are separated from their respective needle introducers such that only part of the first and second devices and the support portion are left in the body, passing from a first exit point in the lower abdominal wall, through the rectus sheath, behind the pubic bone, under the urethra, back behind the pubic bone, back through the rectus sheath and out through a second exit point in the lower abdominal wall.

FIG. 6 shows an implant as described implanted in the body using an obturator foramen procedure. As illustrated in FIG. 6, an incision is made in the wall of the patient's vagina, and a needle introducer, such as a Nottingham needle, to which a first device is attached is inserted through the incision, over to the first obturator foramen and passed through the obturator foramen close to the inferior pubic ramus, through the obturator muscle, and through the perineal skin over a patient's first obturator foramen. Again, as the needle introducer is of small diameter there is no requirement to make a cutaneous incision over the patient's obturator foramen and the needle introducer may be simply pushed through the tissue and thus only create a small puncture in the tissue. The first device is then removed from the needle introducer.

The procedure is repeated such that the second support portion is provided under the urethra with a first device extending out of the first puncture made over the first obturator foramen and a second device extending out of the second puncture made over the second obturator foramen.

Following insertion in the body using either of the above methods, the position of the support and tissue repair devices may be adjusted by pulling on either or both of the ends of the filiform portion which exits the body at the abdomen or above the obturator foramen. Adjustment may be made for around two days following insertion of the devices in the body.

Multiple Tissue Repair Devices Conjoined to Support

The tissue repair device of the present invention may also be used in the treatment of prolapse.

Prolapse can be divided into a number of different categories according to the part of the vagina affected. Prolapse of the anterior (front) vaginal wall (cystourethrocoele) occurs when the bladder and/or the urethra push against and create a bulge in the front wall of the vagina. Prolapse of the posterior (back) vaginal wall can occur if the small intestine (enterocoele) or rectum (rectocoele) loses support and pushes against the back wall of the vagina. Uterine prolapse occurs when the womb drops down into the vagina and vault prolapse can occur in women who have had a hysterectomy.

In the treatment of prolapse, 2, 3, 4, 5, 6, 7, 8, 9, 10 or more tissue repair devices can be conjoined to a support portion. The support portion may be a mesh as described herein and as shown in FIG. 7 and in close up in FIG. 3.

The devices may be attached to the support portion during manufacture of the devices, for example by knitting, or during surgery by for example, polymer rivets or sutures.

The provision of a plurality of tissue repair devices to a support portion, as illustrated in FIG. 7, can be advantageous as they can each be used to provide an additive amount of support to the support portion. The tensile strength required by each tissue repair device can therefore be reduced.

Further, multiple pairs of tissue repair devices conjoined to the support portion are advantageous as they allow the support to be placed over the tissue to be repaired and the tissue repair devices to be more easily inserted around the repair site in a balanced manner to suitably locate the support on the vaginal wall.

In particular embodiments of the implant the support portion is elliptical in shape. This may be advantageous for example in methods to provide support of anterior prolapse, as the support provided will therefore more closely resemble the shape of the tissue of the anterior vaginal wall. Alternatively, the support may be an ellipse or a truncated ellipse.

In alternative embodiments, the support portion is ovoid in shape. This may be advantageous in the treatment of posterior prolapse as the support will more closely resemble the shape of the tissue of the rectocoele.

In a further alternative embodiments the support portion can be bifurcated, H, T, Y, quadrilateral, frog shaped or other desired shapes such that it comprises at least two zones such that in use, the support portion can be positioned on the vaginal wall such that the zones are arranged to pass around the midline to support the vagina. This shape may be advantageous to provide support to tissue on either side. Typically, the support portion can be of a width in the range 3 cm to 12 cm. Typically the tissue repair devices for such an embodiment have a length in the range 4 cm to 20 cm.

In use, the multiple tissue repair devices conjoined to a support portion are inserted by
exposing the segment of vaginal wall and secondary organ prolapse,
placing the appropriately shaped and/or sized support on the vaginal wall,
inserting a first tissue repair device into pelvic paravaginal fibro-fatty tissue, for example the retropubic space, para rectal space, or ischiorectal fossa, and fixing the same into structural tissue or exiting these spaces, through the skin, outside the body,
inserting at least a second tissue repair device into pelvic parvaginal fibro-fatty tissue, for example the retropubic space, para rectal space, or ischiorectal fossa, and fixing the same into structural tissue or exiting these spaces, through the skin, outside the body
inserting any further tissue repair devices into pelvic paravaginal tissue, for example the retropubic space, para rectal space, or ischiorectal fossa, and fixing the same into structural tissue or exiting these spaces, through the skin, outside the body, and
adjusting the tissue repair devices such in the tissue to obtain suitable placement and tension.

As noted above, following insertion in the body, the position of the support and tissue repair devices may be adjusted by pulling on either or both of the ends of the filiform portion which exits the body at the abdomen, above the obturator foramen (perineum) or perineal skin.

Adjustment may be made for around two to three days following insertion of the devices in the body.

As illustrated in FIG. 8 a first tissue repair device 50 with a first end 52 and a second end 54 having a distance between a first end and second end of around 300 mm, (suitably in the range 200 mm to 400 mm) and formed of polypropylene or an absorbable polymer is conjoined at the second end 54 to a short segment of mesh tape 60 of around 120 mm (suitably in the range 80 mm to 140 mm) at a first end 62 of the mesh tape. The second end 64 of the mesh tape 60 is conjoined to a second tissue repair device 70 at one end 72 of the second tissue repair device, which is identical to the first tissue repair device. As illustrated, ends of the tissue repair device not conjoined to the mesh tape, for example the first end 52 of the first tissue repair device 50, can be tapered to a point. This can be advantageous to attach the tissue repair device to a needle introducer to allow insertion of the tissue repair device in the body.

The first tissue repair device is formed from two filaments 55 which have been intertwined, using a warp knit, such that the filaments provide strands 56 with interstices 57 in the range 50 micrometres to 200 micrometres. The strands 56 of intertwined filaments 55 are then further intertwined together such that multiple strands are joined together wherein the spaces 58 between the strands are in the range of 50 micrometres to 200 micrometres. The multiple strands joined together as described. provide a filiform having a longitudinal axis (L) between the first end 52 and the second end 54 and a transverse width (W), perpendicular to the longitudinal axis of about 3 mm. In the illustration provided the thickness of the device cannot be seen, but this is in the range of 20 to 35 micrometres. This thickness of device allows fibroblasts which enter into the interstices between the filaments and the spaces between the strands to extend across the thickness of the device from a first surface to a second surface to hold the device in place in the tissue. In the embodiment illustrated, at the second end 54 of the first tissue repair device 50 the tensioning of the intertwining of the strands is reduced such that the spaces between the strands begin to increase such that the spaces between strands formed by the filaments have a cross-sectional width in the range 1 mm to 10 mm (a) at the support portion of mesh tape 60. The width of the support mesh tape is greater than the tissue repair device and in the embodiment illustrated and is around 11 mm. As illustrated, the interstices between the filaments are still around 50 micrometre to 200 micrometre. Tissue ingrowth at the mesh tape portion can still occur, but the increase in space between the strands will cause this portion of the implant to distort should force be applied along the axis between the first end 62 and second end 64 of the support mesh tape.

As illustrated in FIG. 9, a tissue repair device can be provided with protrusions.

As illustrated in FIG. 9a and FIG. 9b in one embodiment, a projection can be triangular shaped 82 wherein the base 84 of the triangular shaped projection is provided such that it extends perpendicularly from the longitudinal axis of the device 50. In such an embodiment the base of the triangular shaped projection is wider than the width of the tissue repair device at that point, but the thickness of the triangular shaped projection is the same as the device. In such an embodiment the lateral projections from the device will provide for unidirectional movement of the device.

In another embodiment illustrated in FIG. 9c and FIG. 9d, the shaped projection is a rounded protrusion 80 extending from a surface of the tissue repair device 50, but not extending beyond the edges of the device. In such an embodiment the thickness of the device will be greater where the protrusion extends from the surface than at a region of the device adjacent to such a protrusion. However, in such an embodiment the width of the device will not be increased at the protrusion. A protrusion as illustrated in FIG. 9a can provide for a ratcheting movement of the device to be felt by a surgeon as the device, when in use, is pulled through layers of tissue, for example, the endopelvic fascia.

Although various embodiments of the present invention have been described herein, the invention is not limited to these precise embodiments and other changes and modifications may be envisaged by those skilled in the art without departing from the scope of the invention.

1. A tissue repair device comprising a filiform having a longitudinal axis and a transverse width, wherein a force of at least 30 N can be exerted along the longitudinal axis of the device without distortion of the device and at least part of said filiform comprises interstices on a surface of the filiform, the interstices having a cross-sectional width in the range of 50 micrometers to 200 micrometers.

2. The tissue repair device of claim 1, comprising at least one filament wherein said filament is arranged to provide interstices of cross-sectional width in the range 50 micrometers to 200 micrometers.

3. The tissue repair device of claim 2, comprising at least two filaments intertwined together to form a filiform with interstices in the range 50 micrometers to 200 micrometers.

4. The tissue repair device of claim 3, wherein two filaments are arranged to provide strands with interstices in the range 50 micrometers to 200 micrometers between the filaments and the strands of intertwined filaments are further intertwined together such that multiple strands are joined together to form the filiform wherein the spaces between the strands are in the range of 50 micrometers to 200 micrometers.

5. The tissue repair device of claim 1, wherein said filiform is of width in the range 2 mm to 11 mm and of thickness in the range 10 micrometers to 50 micrometers.

6-21. (canceled)

22. An implant comprising the tissue repair device of claim 1 and a support portion.

23. The implant of claim 22, wherein pairs of the tissue repair device are conjoined to the support portion.

24. The implant of claim 23 wherein the support portion is a tape or a mesh.

25. The implant of claim 24, wherein the support portion is a mesh and the mesh comprises spaces between the strands of around 1 to 10 mm.

26-32. (canceled)

33. A method of supporting a defined tissue structure in the body comprising the steps of:
(a) providing at least a first tissue repair device comprising a filiform having a longitudinal axis and a transverse width wherein at least part of said filiform comprises interstices on a surface of the filiform wherein said interstices have a cross-sectional width in the range of 50 micrometers to 200 micrometers and a force of at least 30 N can be exerted along the longitudinal axis of the device without distortion of the device;
(b) fixing a first end of the first device to a first fixing point on a first side of the defined tissue structure to be supported;
(c) fixing a second end of the first device to a second fixing point on a second side of the defined tissue structure to be supported; and
(d) positioning the first device under the defined tissue structure to be supported.

34-37. (canceled)

38. The method of claim 33, further comprising the steps of:
(a) providing a second tissue repair device comprising a filiform having a longitudinal axis and transverse width wherein at least part of said filiform comprises interstices on a surface of the filiform wherein said interstices have a cross-sectional width in the range of 50 micrometres to 200 micrometres and a force of at least 30 N can be exerted along the longitudinal axis of the device without distortion of the device, and a support portion interposed between said first tissue repair device and said second tissue repair device, wherein said support portion is conjoined to one end of the first device and one end of the second device;
(b) inserting a first end of the first tissue repair device through an incision in a body, past a first lateral side of a defined structure to be supported such that that at least part of the first tissue repair device exits the body;
(c) inserting a first end of the second tissue repair device through an incision in the body, past a second lateral side of the defined structure to be supported such that at least part of a second tissue repair device exits the body; and
(d) positioning the support portion interposed between the first and second tissue repair devices adjacent to the defined tissue to be supported such that the support portion provides support to the defined tissue.

39. The method of claim 38, wherein the incision in the body is a vaginal incision, and the defined structure to be supported is a urethra.

40. (canceled)

41. The method of claim 38, further comprising the step of adjusting the position of the support portion under the defined tissue structure to be supported.

42. The method of claim 41 wherein the step of adjusting the position of the support portion under the defined structure to be supported is performed within 72 hours of inserting the device in the body.

43. A method for supporting prolapse in at least one of vagina and uterus, comprising the steps of:
(a) providing at least a first pair and a second pair of tissue repair devices each device comprising a filiform having a longitudinal axis and transverse width wherein at least part of said filiform comprises interstices on a surface of the filiform wherein said interstices have a cross-sectional width in the range of 50 micrometers to 200 micrometers and a force of at least 30 N can be exerted along the longitudinal axis of the device without distortion of the device and a support portion interposed between said first and second pairs of devices, wherein said support portion is conjoined to one end of each of the first and second pairs of devices;
(b) inserting a first end of a first pair of tissue repair devices through a vaginal incision, and through at least one of:
(i) an obturator foramen such that at least part of the tissue repair device exits the body;
(ii) an abdominal wall such that at least part of the tissue repair device exits the body; or
(iii) perineal skin such that at least part of the tissue repair device exits the body;
(c) inserting a first end of a second pair of tissue repair devices through a vaginal incision, and through at least one of:
(i) an obturator foramen such that at least part of the tissue repair device exits the body;
(ii) an abdominal wall such that at least part of the tissue repair device exits the body; or
(iii) perineal skin such that at least part of the tissue repair device exits the body;
(d) positioning the support portion to provide support to at least one of a vaginal wall and a uterus.

44. The method of claim 43, further comprising the step of adjusting the position of the support portion to provide support to at least one of a vaginal wall and a uterus.

45. The method of claim 44, wherein the step of adjusting the position of the support portion is performed within 72 hours of inserting the device in the body.

46. The method of claim 39, wherein the first end of the tissue repair device is further inserted through a first obturator foramen.
